(19) United States
(12) Patent Application Publication
(10) Pub. No.: US 2026/0256832 A1
(43) Pub. Date: Sep. 3, 2026

(54) BIOGLASS COMPOSITION

(71) Applicant: Imam Abdulrahman Bin Faisal University, Dammam (SA)

(72) Inventors: Mohammad Hasan Abu MHAREB, Dammam (SA); Maryam Saeed AL JUMAN, Dammam (SA); Hana Mohammed ALMARRI, Dammam (SA); Mona Yahya ALQAHTANI, Dammam (SA); Norah Hamad ALONIZAN, Dammam (SA)

(73) Assignee: Imam Abdulrahman Bin Faisal University, Dammam (SA)

(21) Appl. No.: 19/068,981

(22) Filed: Mar. 3, 2025

(51) Int. Cl. A61K 33/22; A61K 9/50; C03C 3/064; C03C 4/00; C03C 12/00

(52) U.S. Cl. CPC A61K 33/22; A61K 9/501; C03C 3/064; C03C 4/0014; C03C 12/00; C03C 2204/00

(57) ABSTRACT

A bioglass composition in the form of a particle having a core portion, a crystalline zone and a surface layer. The surface layer is hydroxyapatite, where the crystalline layer is beneath the surface layer and surrounds the core portion. The bioglass composition includes 10 mole percent (mol. %) to 20 mol. % SiO₂, 20 mol. % SrO, 20 mol. % Na₂O, 20 mol. % CaO, and 30 mol. % to 40 mol. % B₂O₃.

### BACKGROUND

### Technical Field

The present disclosure is directed towards bioactive glass material, and more particularly, relates to a bio-glass composition and a bioactivity and biocompatibility thereof.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Presently, civilization relies heavily on synthetic materials interacting with live tissues. The synthetic materials that interact with live tissues are referred to as biomaterials. In particular, a use of synthetic materials in place of natural body parts dates back to several centuries. Among the mummified remains unearthed from historic era, are several that were adorned with gold dental implants. Metals such as bronze and copper were considered the most commonly employed biomaterials [See: Bhatia, Sujata K. Biomaterials for clinical applications]. The application of biomaterials resulted in increased toxicity levels within the human body, which further complicated the situation at the site of implantation. However, biomaterials still remained the most frequently used implant materials until the late 19th century. Aseptic conditions improved in the medical field during the 1980s, and ivory prostheses were manufactured. By the early 19th century, gold capsules had been introduced, laying the groundwork for subsequent advancements. Since then, research is underway for a stable and inert chemical to employ in building gadgets that may come into contact with human tissues [See: Hench, Larry L. Biomaterials: a forecast for the future]. Human life expectancy has increased in recent decades, necessitating a development of innovative biomaterials that may last for an extended period. Biomaterials are desired to possess certain characteristics including biocompatibility, surface activeness, bioactivity, and antibacterial. Further, during selection of a biomaterial, it is critical to rule out factors such as swelling, toxic kinetics reaction, and allergic symptoms in the physiological body system [See: Chen, Qizhi, and George A. Thouas. Metallic implant biomaterials; and Bauer, Sebastian, Patrik Schmuki, Klaus Von Der Mark, and Jung Park. Engineering biocompatible implant surfaces: Part I: Materials and surfaces].

The surface of biomaterials, immediately exposed to live creatures or tissues, is vital for biocompatibility. The surface activity of biomaterials creates challenges that require solutions, in order to maximize the interface between biomaterials and tissues from physical, morphological, and biological perspectives. Biomaterials undergo extensive alterations to attain biocompatibility and surface activity to support specific applications. Currently, enhancing the required properties to enable wounded tissue retrieval is a pressing challenge in regards to biomaterials [See: Lasprilla, Astrid J R, Guillermo A R Martinez, Betânia H. Lunelli, André L. Jardini, and Rubens Maciel Filho. “Poly-lactic acid synthesis for application in biomedical devices-A review].

Long bone fractures are a common result of sports collisions, car accidents, and other stressful events. A cast may repair fractures if the bone is somewhat fractured or displaced. However, in order to precisely align bone pieces in comminuted fractures and open surgery, internal fixation is usually necessary [See: Marsell, Richard, and Thomas A. Einhorn. The biology of fracture healing]. Bone fractures have been treated with bioactive glass substances since the turn of the previous century. Bioactive glass (Bioglass®) was first developed and produced by Larry Hench [See: Hench, Larry L. The story of Bioglass®. Journal of materials science]. When bioactive glasses are immersed in simulated body fluid (SBF), hydroxyapatite (HA) is formed on a surface of the bioglass that was exposed to the SBF. The chemical interaction between HA, the primary component of bone, and the biomaterial results in a strong link between bone tissue and the biomaterial [See: Jones, Julian R. Reprint of: Review of bioactive glass: From Hench to hybrids. Acta biomaterialia 23; and Hoppe, Alexander, Nusret S. Güldal, and Aldo R. Boccaccini. A review of the biological response to ionic dissolution products from bioactive glasses and glass-ceramics.]. Bioactive glasses are biocompatible and may assist in building new and strong bones. Bioactive glass may be further assistive in scaffolding, composites, bone fillers, glass ionomers, toothpaste, dental cement, covering surfaces on metallic implants, and healing wounds and burns [See: Hench, Larry L., Niksa Roki, and Michael B. Fenn. Bioactive glasses: Importance of structure and properties in bone regeneration; and Montazerian, Maziar, and Edgar D. Zanotto. A guided walk through Larry Hench's monumental discoveries. Journal of materials science]. With the discovery of Bioglass®, bioactive silicate glasses have been extensively researched. The glass based on silicate showed promise in biomedical implementations. However, low solubility of the glass based on silicate may render the glass incompatible with biological tissues [See: Huang, Wenhai, Delbert E. Day, Kanisa Kittiratanapiboon, and Mohamed N Rahaman. Kinetics and mechanisms of the conversion of silicate (45S5), borate, and borosilicate glasses to hydroxyapatite in dilute phosphate solutions. Journal of materials science; and Brown, Roger F., Mohamed N. Rahaman, Agatha B. Dwilewicz, Wenhai Huang, Delbert E. Day, Yadong Li, and B. Sonny Bal. Effect of borate glass composition on its conversion to hydroxyapatite and on the proliferation of MC3T3-E1 cells. Journal of biomedical materials research]. 45S5, a silicate bioactive glass with the ingredients (in weight percentage) including 45 wt. % SiO₂, 24.5 wt. % Na₂O, 24.5 wt. % CaO, and 6 wt. % P₂O₅, has been produced. 45S5 is used in a vast majority of biological research and implementations. Lately, 13-93, a silicate bioactive glass comprised of 53 wt. % SiO₂, 6 wt. % Na₂O, 12 wt. % K₂O, 5 wt. % MgO, 20 wt. % CaO, and 4 wt. % P₂O₅, has gained interest for its possible medical applications [See: Brink, Maria, Tia Turunen, Risto-Pekka Happonen, and Antti Yli-Urpo. Compositional dependence of bioactivity of glasses in the system Na₂O—K₂O—MgO—CaO—B₂O₃—P₂O₅—SiO₂. Journal of biomedical materials research]. The food and drug administration (FDA) of the United States approved the in vivo use of two types of bioactive glass including 13-93 glasses and 45S5 glasses. Furthermore, when exposed to SBF, 13-93 and 45S5 glasses gradually and partially change into HA. Glass-forming borate composites are an alternative that is now being evaluated for potential use in biomedical applications. In particular, bioactive glasses based on borate were recently developed for possible biological implementations [See: Rahaman, Mohamed N., Roger F. Brown, B. Sonny Bal, and Delbert E. Day. Bioactive glasses for nonbearing applications in total joint replacement]. Due to low chemical stability borate glasses, certain specific borate glasses convert to HA in a liquid phosphate solution as a SBF more quickly and thoroughly than certain other alternate bioactive glasses. Borate glasses showed higher bioactivity than silicate glass due to a limited chemical stability of the borate glasses, quick breakdown in biological fluids, and higher HA conversion rate. Researchers have explored the impact of partly or wholly substituting a B₂O₃ ratio for the SiO₂ ratio in the 45S5 glass on mechanism and kinetics of converting glass particles to HA. It was found that the transformation rate increased for the glass with rising B₂O₃ content. Recently, porous 3D scaffolds of B₂O₃-containing glasses with specified compositions were produced, and the scaffolds showed a desirable transformation to HA in an SBF in vitro [See: Baghbani, Fatemeh, Fathollah Moztarzadeh, Masoud Mozafari, Majid Raz, and Hamideh Rezvani. Production and characterization of an Ag- and Zn-doped glass ceramic material and in vitro evaluation of its biological effects; and Mariappan, C. R., and N. Ranga. Influence of silver on the structure, dielectric and antibacterial effect of silver doped bioglass-ceramic nanoparticles]. Nevertheless, the effect of the B₂O₃ content of the glasses on scaffold deterioration has not been adequately examined. Furthermore, bacterial infections, whether acquired in the hospital or due to surgical treatment failures, complicate using glasses to treat bone fractures. Hence, the antibacterial features of bioactive glass may be improved for better biocompatibility

Accordingly, one object of the present disclosure is to provide a bioglass composition in the form of a particle, that may circumvent the drawbacks, such as, low biocompatibility, limited efficacy, less stability, lower antiseptic activity, and high economical factors, of materials known in the art.

### SUMMARY

In an exemplary embodiment, a bioglass composition in the form of a particle is described. The bioglass composition has a core portion, a crystalline zone and a surface layer. The surface layer is hydroxyapatite, where the crystalline layer is beneath the surface layer and surrounds the core portion. The bioglass composition includes 10 mole percent (mol. %) to 20 mol. % SiO₂, 20 mol. % SrO, 20 mol. % Na₂O, 20 mol. % CaO, and 30 mol. % to 40 mol. % B₂O₃.

In some embodiments, the composition in the form of a pressed pellet, includes a plurality of particles of the composition, the particles have a longest dimension in a range from 0.5 mm to 1000 mm.

In some embodiments, the pressed pellet has a circular form with a circumference in a range from 0.5 to 100 mm and a thickness in a range from 0.1 to 100 mm.

In some embodiments, the surface layer includes hydroxyapatite crystals formed by immersion in a simulated body fluid.

In some embodiments, hydroxyapatite crystals are in the form of a uniform layer and are formed by 21 days immersion in the simulated body fluid.

In some embodiments, the bioglass composition includes 10 mol. % SiO₂, 20 mol, % SrO, 20 mol. % Na₂O, 20 mol. % CaO, and 40 mol. % B₂O₃ and has a weight loss greater than or equal to 8 percent after 7 days of immersion in a simulated body fluid.

In some embodiments, the bioglass composition has a weight loss greater than or equal to 10 percent after 15 days of immersion in simulated body fluid.

In some embodiments, the bioglass composition has a weight loss greater than or equal to 12 percent after 21 days of immersion in simulated body fluid.

In some embodiments, the bioglass composition includes 20 mol. % SiO₂, 20 mol. % SrO, 20 mol. % Na₂O, 20 mol. % CaO, and 30 mol. % B₂O₃ and has a weight loss greater than or equal to 10 percent after 7 days of immersion in simulated body fluid.

In some embodiments, the bioglass composition has a weight loss greater than or equal to 13 percent after 15 days of immersion in simulated body fluid.

In some embodiments, the bioglass composition has a weight loss greater than or equal to 15 percent after 21 days of immersion in simulated body fluid.

In some embodiments, x-ray diffraction shows evidence of calcium deficient hydroxyapatite formation by the presence of a peak at around 26° after 21 days of immersion in simulated body fluid.

In some embodiments, the bioglass composition has an antibacterial activity of greater than or equal to 23 mm for Staphylococcus epidermidis when measured in a direct contact incubation test.

In some embodiments, the bioglass composition has an antibacterial activity of greater than or equal to 24 mm for Staphylococcus aureus when measured in a direct contact incubation test.

In some embodiments, the bioglass composition has an antibacterial activity of greater than or equal to 21 mm for Escherichia coli when measured in a direct contact incubation test.

In some embodiments, the bioglass composition has an antibacterial activity of greater than or equal to 22 mm for Shigella flexneri when measured in a direct contact incubation test.

In some other embodiments, the bioglass composition has an antibacterial activity of greater than or equal to 21 mm for Staphylococcus epidermidis when measured in a direct contact incubation test.

In some embodiments, the bioglass composition has an antibacterial activity of greater than or equal to 20 mm for Staphylococcus aureus when measured in a direct contact incubation test.

In some embodiments, the bioglass composition has an antibacterial activity of greater than or equal to 21 mm for Escherichia coli when measured in a direct contact incubation test.

In some embodiments, the bioglass composition has an antibacterial activity of greater than or equal to 20 mm for Shigella flexneri when measured in a direct contact incubation test.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is an optical image depicting a pattern of fabricated glass sample SO, S10, and S20, post processing as pellets, according to certain embodiments.

FIG. 2A illustrates an X-ray diffraction (XRD) spectra of the sample SO, according to certain embodiments.

FIG. 2B illustrates the XRD spectra of the sample S10, according to certain embodiments.

FIG. 2C illustrates the XRD spectra of the sample S20, according to certain embodiments.

FIG. 3 illustrates the XRD spectra of SO, S10, and S20 glass samples after immersion in simulated body fluid (SBF) for 21 days, according to certain embodiments.

FIG. 4A illustrates a Fourier transform infrared (FTIR) absorption spectra of the sample SO, before and after immersion in SBF, according to certain embodiments.

FIG. 4B illustrates the FTIR absorption spectra of the sample S10, before and after immersion in SBF, according to certain embodiments.

FIG. 4C illustrates the FTIR absorption spectra of the sample S20, before and after immersion in SBF, according to certain embodiments.

FIG. 5A illustrates a scanning electron microscopy (SEM) image of a surface of the sample SO, before immersion in SBF, according to certain embodiments.

FIG. 5B illustrates the SEM image of the surface of the sample SO, after 7 days of immersion in SBF, according to certain embodiments.

FIG. 5C illustrates the SEM image of the surface of the sample SO, after 15 days of immersion in SBF, according to certain embodiments.

FIG. 5D illustrates the SEM image of the surface of the sample SO, after 21 days of immersion in SBF, according to certain embodiments.

FIG. 5E illustrates the SEM image of a surface of the sample S10, before immersion in SBF, according to certain embodiments.

FIG. 5F illustrates the SEM image of the surface of the sample S10, after 7 days of immersion in SBF, according to certain embodiments.

FIG. 5G illustrates the SEM image of the surface of the sample S10, after 15 days of immersion in SBF, according to certain embodiments.

FIG. 5H illustrates the SEM image of the surface of the sample S10, after 21 days of immersion in SBF, according to certain embodiments.

FIG. 5I illustrates the SEM image of a surface of the sample S20, before immersion in SBF, according to certain embodiments.

FIG. 5J illustrates the SEM image of the surface of the sample S20, after 7 days of immersion in SBF, according to certain embodiments.

FIG. 5K illustrates the SEM image of the surface of the sample S20, after 15 days of immersion in SBF, according to certain embodiments.

FIG. 5L illustrates the SEM image of the surface of the sample S20, after 21 days of immersion in SBF, according to certain embodiments.

FIG. 6A illustrates a graph depicting boron (B) ion concentration for the three glass samples S0, S10, and S20 at a plurality of immersion time, according to certain embodiments.

FIG. 6B illustrates a graph depicting silicon (Si) ion concentration for the three glass samples S0, S10, and S20 at the plurality of immersion time, according to certain embodiments.

FIG. 7A is an optical image of a petri dish depicting an antibacterial activity of the three glass samples S0, S10, and S20, against Staphylococcus epidermidis (ATCC 12228), according to certain embodiments.

FIG. 7B is an optical image of a petri dish depicting the antibacterial activity of the three glass samples S0, S10, and S20, against Escherichia coli (ATCC 25922), according to certain embodiments.

FIG. 7C is an optical image of a petri dish depicting the antibacterial activity of the three glass samples S0, S10, and S20, against Staphylococcus aureus (ATCC 29213), according to certain embodiments.

FIG. 7D is an optical image of a petri dish depicting the antibacterial activity of the three glass samples S0, S10, and S20, against Shigella flexneri (ATCC 12022), according to certain embodiments.

FIG. 8A is a high resolution B 1s spectra for the three glass samples S0, S10, and S20, according to certain embodiments.

FIG. 8B is a curve fitted B 1s spectrum for the glass sample SO, according to certain embodiments.

FIG. 9A is a high resolution O 1s spectra for the three glass samples S0, S10, and S20, according to certain embodiments.

FIG. 9B is a curve fitted O 1s spectrum for the glass sample SO, depicting BO and NBO contributions, according to certain embodiments.

FIG. 10A is a high resolution Na 1s spectra for the three glass samples S0, S10, and S20, according to certain embodiments.

FIG. 10B is a curve fitted Na 1s spectrum for the glass sample S20, depicting a single contribution, according to certain embodiments.

FIG. 11A is a high-resolution Ca 2p core level spectra for the three glass samples S0,

S10, and S20, depicting a standard spin-orbit doublet of the 2p level, according to certain embodiments.

FIG. 11B is a curve fitted Ca 2p spectrum for the glass sample SO, depicting a single contribution to the 2p_{1/2} and a single contribution to the 2p_{3/2} peaks, according to certain embodiments.

FIG. 12A is a high-resolution Sr 3d core level spectra for the three glass samples S0, S10, and S20, depicting a standard spin-orbit doublet of the 3d level, according to certain embodiments.

FIG. 12B is a curve fitted Sr 3d spectrum for the glass sample S20, depicting a single contribution to the 3d_{3/2} and a single contribution to the 3d_{5/2} peaks, according to certain embodiments.

FIG. 13A is a high-resolution Si 2p core level spectra for the three glass samples S0, S10, and S20, according to certain embodiments.

FIG. 13B is a curve fitted Si 2p spectrum for the glass sample S10, depicting a spin-orbit contribution to the main peak, according to certain embodiments.

### DETAILED DESCRIPTION

When describing the present disclosure, the terms used are to be construed in accordance with the following definitions, unless a context dictates otherwise.

Embodiments of the present invention will now be described more fully hereinafter with reference to the accompanying drawings wherever applicable, in that some, but not all, embodiments of the disclosure are shown.

In the drawings, like reference numerals designate identical or corresponding parts throughout the several views. Further, as used herein, the words ‘a,’ ‘an’ and the like generally carry a meaning of ‘one or more,’ unless stated otherwise.

Furthermore, the terms ‘approximately,’ ‘approximate,’ ‘about,’ and similar terms generally refer to ranges that include the identified value within a margin of 20%, 10%, or preferably 5%, and any values therebetween.

As used herein, the term ‘bioactive glass’ refers to a glass composition that interacts with biological systems to promote tissue regeneration, particularly in the contexts of bone and dental repair. The bioactive glass is capable of forming a hydroxyapatite (HA) layer on its surface when immersed in physiological solutions, such as simulated body fluid (SBF), which is indicative of its ability to bond with bone tissue.

As used herein, the term ‘simulated body fluid (SBF)’ refers to an aqueous solution that mimics the ionic composition and pH of human blood plasma. SBF is used to assess the bioactivity of materials by simulating the conditions under which biomaterials would interact with the human body in vivo.

As used herein, the term ‘hydroxyapatite (HA)’ refers to a naturally occurring calcium phosphate mineral that is the primary inorganic component of bone and teeth. In the context of the bioactive glass, the formation of HA on the surface of the material during immersion in SBF is a key indicator of its biocompatibility and its potential to support bone and dental tissue healing.

As used herein, the term ‘crystalline zone’ refers to the area beneath the surface layer of the bioactive glass, which remains in a more ordered, crystalline state compared to the surface layer. This crystalline zone plays a significant role in the material's overall bioactivity and its ability to form HA crystals.

As used herein, the term ‘surface layer’ refers to the outermost layer of the bioactive glass, which becomes enriched with hydroxyapatite (HA) crystals upon immersion in simulated body fluid. This surface layer is essential for the bioactive glass's ability to integrate with biological tissues, particularly bone and dental tissues.

As used herein, the term ‘core portion’ refers to the central region of the bioactive glass particle, which is surrounded by the crystalline zone and the surface layer. The core portion contains the primary glass material that undergoes degradation and transformation when exposed to simulated body fluid.

As used herein, the term ‘X-ray diffraction (XRD)’ refers to an analytical technique used to study the crystallographic structure of materials. XRD is employed in the present disclosure to identify the formation of calcium-deficient hydroxyapatite on the bioactive glass after immersion in simulated body fluid. A characteristic peak around 26° in the XRD pattern signifies the presence of hydroxyapatite.

As used herein, the term ‘scanning electron microscopy (SEM)’ refers to a high-resolution imaging technique that uses electron beams to visualize the surface structure of a material. In the present disclosure, SEM is used to observe the morphological changes in the bioactive glass, such as the formation of hydroxyapatite crystals over time.

As used herein, the term ‘Fourier-transform infrared (FTIR) spectroscopy’ refers to an analytical technique used to obtain the infrared spectrum of a material, which provides information about the chemical bonds and functional groups present in the sample. FTIR is used in this disclosure to confirm the presence of hydroxyapatite on the bioactive glass surface and to analyze the chemical changes during the immersion process.

As used herein, the term ‘weight loss’ refers to the decrease in mass of the bioactive glass during immersion in simulated body fluid, indicating the material's rate of degradation. Weight loss is an important measure of the bioactive glass's ability to gradually release ions and interact with the surrounding biological environment, promoting tissue healing. In the present disclosure, the bioactive glass shows specific weight loss percentages after 7, 15, and 21 days of immersion, demonstrating its gradual degradation over time.

As used herein, the term ‘antibacterial activity’ refers to the ability of a material to inhibit the growth or kill bacteria. The antibacterial activity of the bioactive glass is measured using a direct contact incubation method, where the material is tested against different bacterial strains. The size of the inhibition zone (measured in millimeters) around the bioactive glass indicates its effectiveness in preventing bacterial growth.

As used herein, the term ‘direct contact incubation test’ refers to an experimental method used to assess the antibacterial activity of a material by placing the material in direct contact with bacterial cultures and observing the inhibition of bacterial growth. The size of the inhibition zone surrounding the material is measured to determine its antibacterial effectiveness.

According to an aspect of the present disclosure are directed to a bioactive glass that incorporates borate and silicate for potential use as a biomaterial. The glass is evaluated for its bioactivity and antibacterial properties by immersing it in simulated body fluid (SBF) for various time periods (0, 7, 15, and 21 days). The morphological and structural changes are studied using techniques such as X-ray diffraction (XRD), scanning electron microscopy (SEM), and Fourier-transform infrared (FTIR) spectroscopy. These analyses revealed the formation of a hydroxyapatite layer, confirming the glass's biocompatibility and ability to promote bone and dental healing. The results suggest that the developed bioactive glass has promising potential for biomedical applications.

A bioglass composition in the form of a particle has a core portion, a crystalline zone and a surface layer. In one or more embodiments, the core portion is the bioglass according the compositions shown below, and may be designed to have specific characteristics such as high density or porosity, which can influence how the material degrades over time or interacts with biological tissues. In some embodiments, the crystalline zone may vary in thickness and composition depending on the processing conditions or immersion time. It may contain compounds such as calcium-deficient hydroxyapatite or other calcium phosphate phases. In one or more embodiments, the crystalline zone is formed on bioglass composition during exposure to SBF or similar conditions. Therefore, the crystalline zone may be variable in its specific make-up and morphology, which will depend on the immersion conditions. The crystalline layer is beneath the surface layer and surrounds the core portion. In one or more embodiments, the surface layer is hydroxyapatite. The thickness of the hydroxyapatite layer may vary due to immersion conditions.

In some embodiments, the surface layer may be designed to form a uniform, dense coating of hydroxyapatite to promote better interaction with bone tissue or to provide antibacterial properties.

The bioglass composition includes 10-20 mol % SiO₂, 20 mol % SrO, 20 mol % Na₂O, 20 mol % CaO, and 30-40 mol % B₂O₃. In one embodiment, the bioglass composition includes 10-20 mol % SiO₂, 20 mol % SrO, 20 mol % Na₂O, 20 mol % CaO, and 30 mol % B₂O₃. In one embodiment, the bioglass composition includes 10-20 mol % SiO₂, 20 mol % SrO, 20 mol % Na₂O, 20 mol % CaO, and 20 mol % B₂O₃. In one embodiment, the bioglass composition includes 10 mol % SiO₂, 20 mol % SrO, 20 mol % Na₂O, 20 mol % CaO, and 40 mol % B₂O₃.

In some embodiments, the bioglass composition is in the form of a pressed pellet, including a plurality of particles of the composition. The particles have the longest dimension in a range from 0.5 to 1000 mm. The pressed pellet has a circular form with a circumference in a range from 0.5 to 100 mm and a thickness in a range from 0.1 to 100 mm. The geometry of the pressed pellet may vary and may include, but are not limited to, polygonal, triangular, and rectangular.

The surface layer includes hydroxyapatite crystals, preferably in a uniform layer, and are formed by immersion in a simulated body fluid for greater than 7 days, preferably 15 days, and more preferably for 21 days.

In some embodiments, the bioglass composition including 10 mol % SiO₂, 20 mol % SrO, 20 mol % Na₂O, 20 mol % CaO, and 40 mol % B₂O₃ has a weight loss greater than or equal to 8 percent, preferably 8.971% after 7 days of immersion in a simulated body fluid; a weight loss greater than or equal to 10 percent, preferably 11.850%, after 15 days of immersion in simulated body fluid; and a weight loss greater than or equal to 12 percent, preferably 12.577% after 21 days of immersion in simulated body fluid.

In some embodiments, the bioglass composition, including 20 mol % SiO₂, 20 mol % SrO, 20 mol % Na₂O, 20 mol % CaO, and 30 mol % B₂O₃, has a weight loss greater than or equal to 10 percent, preferably 10.505% after 7 days of immersion in simulated body fluid; a weight loss greater than or equal to 13 percent, preferably 13.760% after 15 days of immersion in simulated body fluid; and a weight loss greater than or equal to 15 percent, preferably 15.800% after 21 days of immersion in simulated body fluid.

The X-ray diffraction shows evidence of calcium-deficient hydroxyapatite formation by a peak at around 26° after 21 days of immersion in simulated body fluid.

In some embodiments, the bioglass composition has an antibacterial activity against gram-positive and gram-negative bacteria. In an embodiment, the bioglass composition has an antibacterial activity of greater than or equal to 21 mm, more preferably 23 mm for Staphylococcus epidermidis when measured in a direct contact incubation test. In one embodiment, the bioglass composition has an antibacterial activity of 26.82 mm for Staphylococcus epidermidis when measured in a direct contact incubation test. In some embodiments, the bioglass composition has an antibacterial activity of 23.54 mm for Staphylococcus epidermidis when measured in a direct contact incubation test. In some embodiments, the bioglass composition has an antibacterial activity of 21.63 mm for Staphylococcus epidermidis when measured in a direct contact incubation test.

The bioglass composition has an antibacterial activity of greater than or equal to 20 mm, more preferably 24 mm for Staphylococcus aureus when measured in a direct contact incubation test. In some embodiments, the bioglass composition has an antibacterial activity of 27.07 mm for Staphylococcus aureus when measured in a direct contact incubation test. In some embodiments, the bioglass composition has an antibacterial activity of 24.94 mm for Staphylococcus aureus when measured in a direct contact incubation test. In some embodiments, the bioglass composition has an antibacterial activity of 20.28 mm for Staphylococcus aureus when measured in a direct contact incubation test.

The bioglass composition has an antibacterial activity of greater than or equal to 21 mm for Escherichia coli when measured in a direct contact incubation test. In some embodiments, the bioglass composition has an antibacterial activity of 29.86 mm for Escherichia coli when measured in a direct contact incubation test. In some embodiments, the bioglass composition has an antibacterial activity of 22.04 mm for Escherichia coli when measured in a direct contact incubation test. In some embodiments, the bioglass composition has an antibacterial activity of 21.65 mm for Escherichia coli when measured in a direct contact incubation test.

The bioglass composition has an antibacterial activity of greater than or equal to 20 mm, more preferably 22 mm for Shigella flexneri when measured in a direct contact incubation test. In some embodiments, the bioglass composition has an antibacterial activity of 30.46 mm for Shigella flexneri when measured in a direct contact incubation test. In some embodiments, the bioglass composition has an antibacterial activity of 23.05 mm for Shigella flexneri when measured in a direct contact incubation test. In some embodiments, the bioglass composition has an antibacterial activity of 20.61 mm for Shigella flexneri when measured in a direct contact incubation test.

### EXAMPLES

The following examples demonstrate a bioglass composition and a bioactivity and biocompatibility analysis thereof. The examples are provided solely for illustration and are not to be construed as limitations of the present disclosure, as many variations thereof are possible without departing from the spirit and scope of the present disclosure.

### Example 1: Materials and Methods (Glass Fabrication)

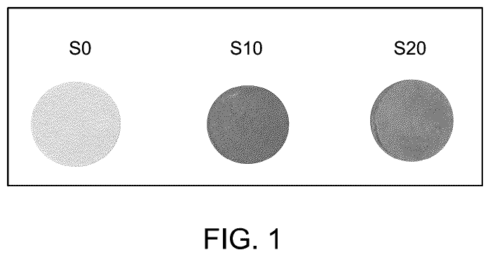

In the present disclosure, three bioactive glasses based on borate and silicate glass were fabricated using a melt-quench technique. A plurality of raw materials employed for the glass production included, boron oxide (B₂O₃), sodium oxide (Na₂O), calcium oxide (CaO), and strontium oxide (SrO). The aforementioned oxides were mixed carefully based on the molar composition ratio, as listed in Table 1. The mixture of the oxides was inserted in an electrical furnace at a temperature range of 1100° C. to 1450° C. based on the SiO₂ ratio. The mixture was constantly stirred, to affirm the homogeneity and discharge gas bubbles, during the synthesization. Further, annealing was employed which included the poured mixture in another electrical furnace at around 400° C. for 5 hours. The furnace temperature was lowered to reach room temperature five hours thereafter. Furthermore, the glass was ground and pressed as a disc for further evaluations using different examination techniques. The synthesized glass samples are shown in FIG. 1.

TABLE 1
Density values and molar composition
ratio of all evaluated samples

Composition ratio (mol. %)

| Glass codes | SiO₂ | SrO | NaO₂ | CaO | B₂O₃ |
|---|---|---|---|---|---|
| S0 | 0 | 20 | 20 | 20 | 40 |
| S10 | 10 | 20 | 20 | 20 | 30 |
| S20 | 20 | 20 | 20 | 20 | 20 |

### Example 2: Biocompatibility of the Glasses (In-Vitro)

The simulated body fluid from Biochemazone company was used to evaluate the biocompatibility of the glass system synthesized herein. A plurality of physicochemical instrument, like X-ray diffraction (XRD), Fourier transform infrared (FTIR) spectrometer, and scanning electron microscope (SEM), were used for the examinations conducted with respect to the present disclosure.

### Example 3: XRD

In general, XRD is used for determining a structure of solid and crystallinity of substances. The amorphous conduct of the produced glasses was evaluated via Shimadzu XRD-7000 equipment (Cu, λ=1.504 Å) at 2θ range of 10° to 90° and a scan rate of 2°/min. The biocompatibility of synthesized glass was further evaluated by determining the crystalline phases created by immersion in simulated body fluid (SBF).

### Example 4: FTIR Spectroscopy

FTIR spectroscopy is used for evaluating structural groups in amorphous and crystalline structure. FTIR collects data to identify the chemical and vibrational bonding between substances. Glass functional groups were investigated before and after SBF immersion using a PerkinElmer Spectrum 100 (Waltham, MA, USA) instrument equipped with attenuated total reflection (ATR-FTIR) for a wavenumber range of about 400 cm⁻¹ to about 2000 cm⁻¹.

### Example 5: SEM

A FEI, Inspect S50 scanning electron microscope operates was employed for the SEM evaluations. The aforementioned SEM was operated at an accelerating voltage of 20 kilovolts (kV) to examine the surface morphology. The biocompatibility of glass samples was measured by exploring the crystalline formation of glasses after immersion in SBF.

### Example 6: Bioactivity of Glasses

The bioactivity (degradation) of the three glass systems and associated transformation to HA was assessed as a function of the sample immersion period in an SBF with an initial pH of 6.1 at a temperature of about 37° C.

### Example 7: Degradation

In order to track the conversion kinetics over time, weight loss that occurs along with the degradation and conversion process was utilized. For every conversion experiment, a ratio of about 0.5 grams (g) of the synthesized glass disc per 15 mL of SBF were used. The discs were removed from the SBF at predetermined intervals, dried at 100° C., and weighed. The following relation was employed to calculate the weight loss (DWₜ),

$${DW}_{t} = \frac{( {W}_{0} - {W}_{t} )}{{W}_{0}} \times 100$$

where ‘Wₜ’ is the mass at time ‘t’, and ‘W₀’ is a starting mass for the disc under analysis. The solution temperature was reduced to room temperature followed by a pH determination using a pH meter.

### Example 8: Inductively Coupled Plasma Mass Spectrometry (ICP-MS)

Ion release concentrations were performed using an ICPE-9800 apparatus. On the glass series, ion release was measured every 3, 7, and 14 days. Further, 15 mL of deionized water and 0.15 g of glass were combined, and the samples were rotated to prevent settling while the mixture was incubated at 37° C. The samples were centrifuged and the supernatant material was used for analysis. The following lines for a plurality of elements were examined, B (208.959 nm), Sr (216.596 nm), Na (330.232 nm), Si (288.158 nm), and Ca (422.673 nm).

### Example 9: Antibacterial Test

According to the present disclosure, antibacterial activity of the synthesized glass samples was assessed using direct contact against four pathogenic bacterial strains involving two gram-positive bacteria and two gram-negative bacteria, namely, Escherichia coli (ATCC 259220, Staphylococcus epidermidis (ATCC 122280, Staphylococcus aureus (ATCC 29213), and Shigella flexneri (ATCC 12022). Tested strains were inoculated on Muller-Hinton agar (MHA) plates composed of beef extract (2.0 g/l), casein hydrolysate (17.5 g/l), starch (1.5 g/l) and bacteriological agar (17 g/l). The culture media was sanitized by autoclaving at a temperature of about 121° C. for 15 minutes, and its pH was adjusted to 7.0. The medium was poured onto Petri dishes, after solidification, bacterial lawns were swabbed on the surface of the medium at right angles. Lawns were taken from bacterial suspensions involving 108 colony-forming units per milliliters (CFU/mL). Three discs, each measuring about 14 mm, with varying concentrations of about 0%, 10%, and 20% of silicon, 40%, 30%, and 20% of boron and a fixed mixture of other chemical components such as Ca, Sr, and Na were transferred on the surface of agar medium. Furthermore, the plate cultures were incubated for 24 hours at 37° C. The antimicrobial response was defined by measuring the diameter of inhibition zones (in millimeters) around the synthesized discs [See: Tenover, F. C. Antimicrobial susceptibility testing. Schmidt, T. M. Edition: Encyclopaedia of microbiology (fourth edition), Academic Press, Elsevier, Cambridge].

### Example 10: X-Ray Photoelectron Spectroscopy (XPS)

A Thermo-Scientific Escalab 250 Xi XPS spectrometer was utilized to evaluate the chemical structure, bonding environments and oxidation states of the elements involved in the three samples. Further the three glass samples were examined using an Al—Kα monochromatic X-ray source. In order to irradiate glasses and offset the charging impacts, a flood gun was employed. The samples were subjected to an argon (Ar⁺) ion bombardment for 20 seconds at an accelerating potential of 3.0 kV in order to eliminate surface contaminants including carbon and oxygen. In order to determine the glass compositions, a survey scan was acquired. The XPS spectra of O 1s, C 1s, Sr 3d, B 1s, Na 1s, Si 2p, and Ca 2p were acquired at a high resolution of around 0.1 eV. The base pressure was about 1×10-7 millibar (mbar) during the ion bombardment. Using the adventitious C 1s at a binding energy of 284.8 eV, all spectra were calibrated. An updated version of the Avantage software was utilized to match the experimental data, which was integrated into the XPS apparatus used herein.

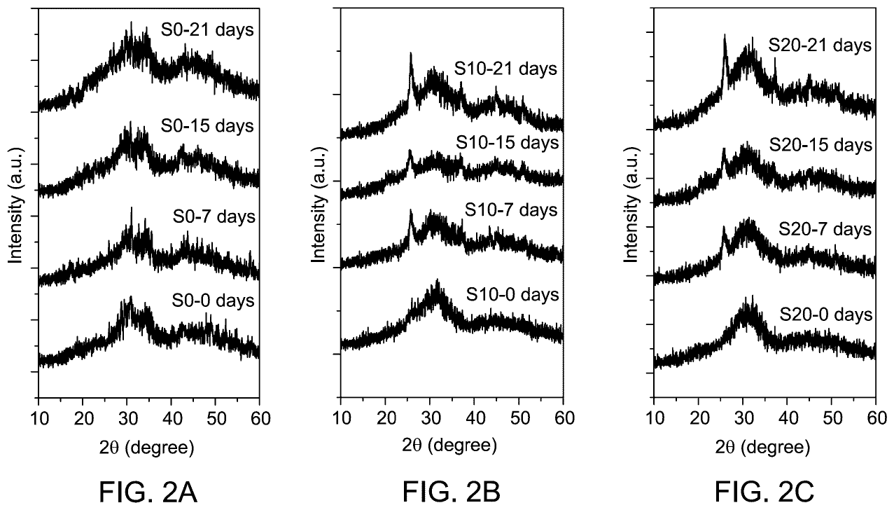

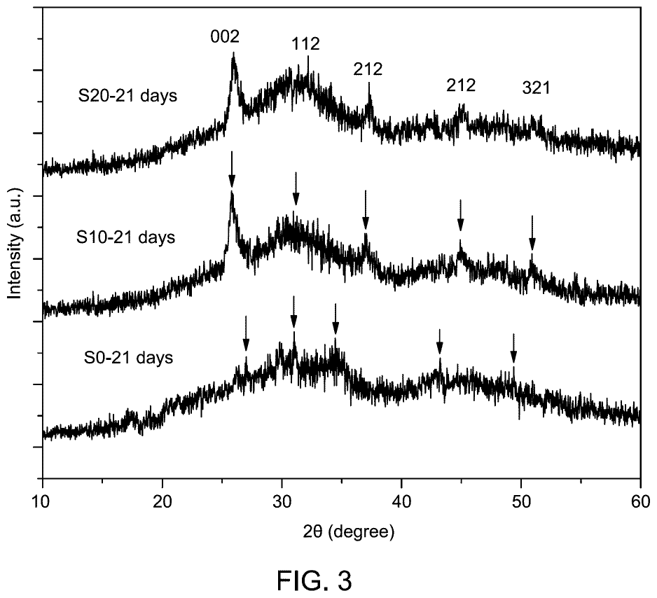

XRD patterns of the three glass samples obtained from the melt quench process, before and after immersion in SBF for a period of 7 days, 15 days, and 21 days for samples S0 (0% silicon, 40% boron), S10 (10% silicon, 30% boron), and S20 (20% silicon, 20% boron), are shown in FIGS. 2A-2C. The amorphous nature of all samples before immersion may be noted, as evidenced by the absence of sharp crystalline peaks. Post immersion in SBF, the XRD spectrum for the S0 sample did not show any crystalline peaks after immersion in SBF for the time intervals including 7 days, 15 days, and 21 days, as shown in FIG. 2A, indicating non-formation hydroxyapatite (HA) in the S0 sample. Further, as can be seen from FIG. 2B-2C, a novel phase emerged for samples S10 and S20, progressively crystallizing from 7 days and reaching clarity up to 21 days, with the location of a peak at 26°, 32°, 38°, 46°, and 51°, signifying the genesis of hydroxyapatite. The XRD pattern of calcium-deficient hydroxyapatite (CDHAP) may exhibit similar peaks at 2θ values of around 26°, 32°, 38°, 46° and 51° which correspond to the (002), (112), (212), (400), and (321) crystal planes of the S10 and S20 samples, as shown in FIG. 3, respectively [See: Sawada, Michika, Kandi Sridhar, Yasuharu Kanda, and Shinya Yamanaka. “Purev hydroxyapatite synthesis originating from amorphous calcium carbonate”, and El Boujaady, H., M. Mourabet, A. El Rhilassi, M. Bennani-Ziatni, R. El Hamri, and A. Taitai. “Adsorption of a textile dye on synthesized calcium deficient hydroxyapatite (CDHAP): Kinetic and thermodynamic studies”].

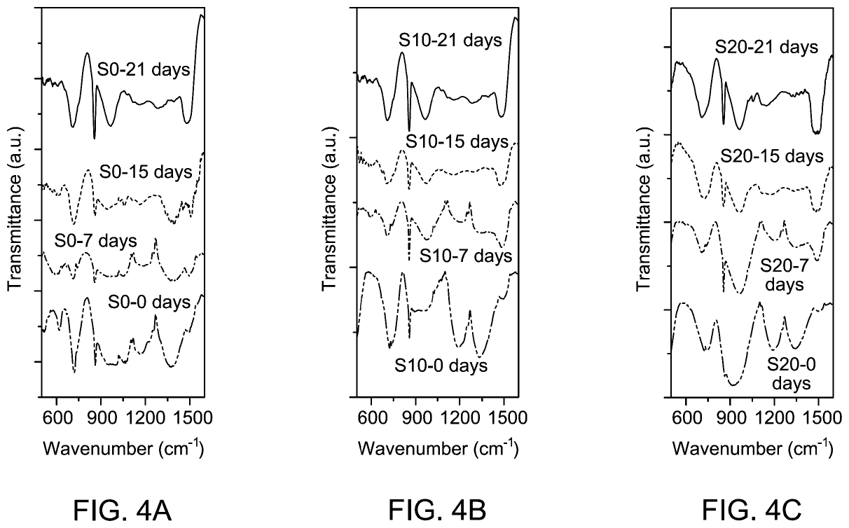

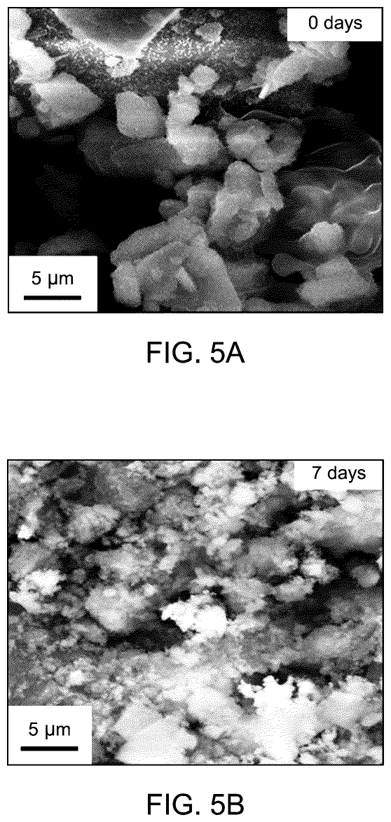

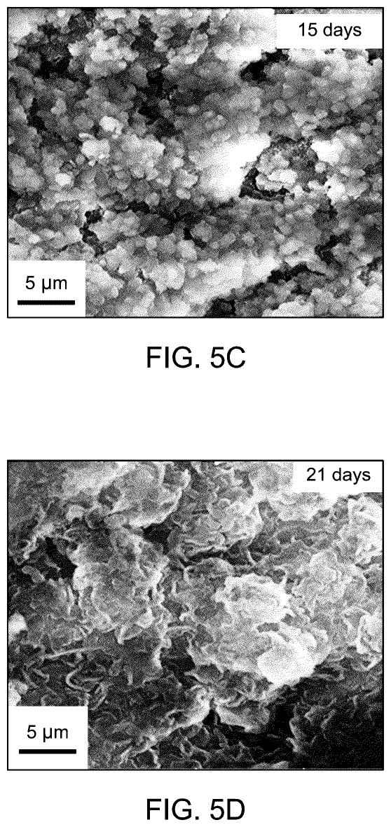

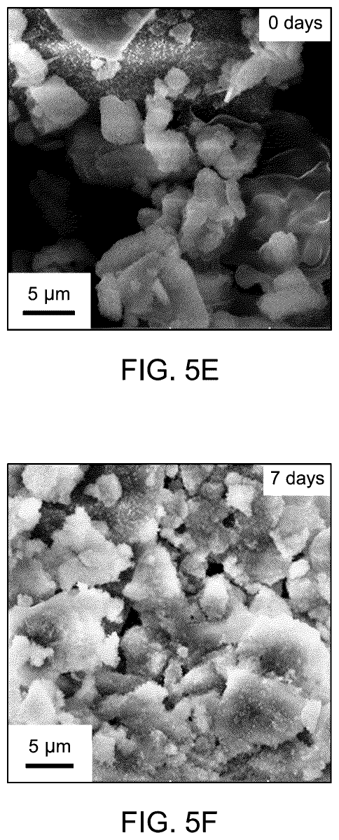

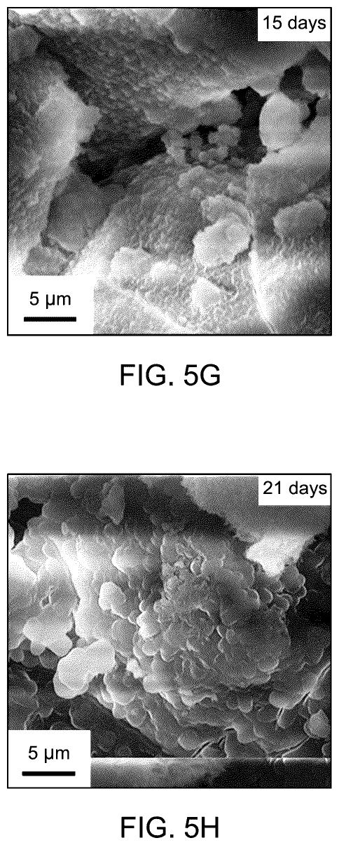

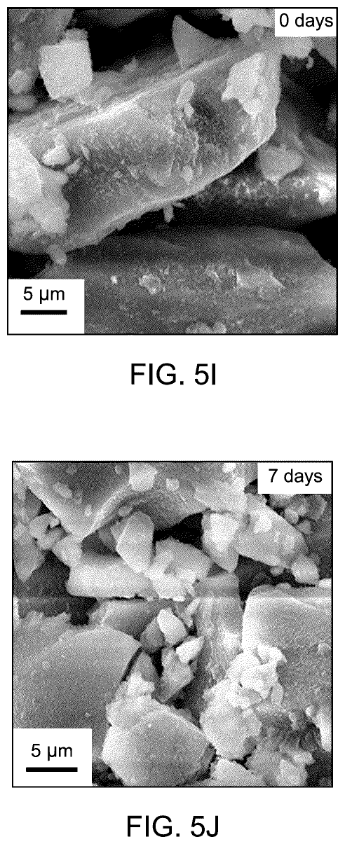

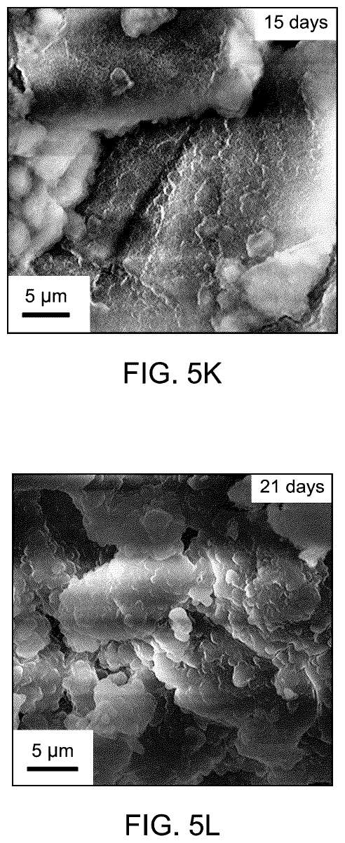

FIGS. 4A-5C shows the FTIR spectra of the three glass samples, in a wavenumber range of 1600 cm⁻¹ to 500 cm⁻¹, at room temperature. As can be seen from FIGS. 4A-5C, the FTIR results are indicated for the samples S0, S10, and S20, before and after immersion in SBF for 7 days, 15 days, and 21 days. The major differences in the FTIR results for the samples S0, S10, and S20, lie in the peak intensities, shifts, and appearances/disappearances. For instance, in sample S0 before immersion, the bands observed at around 618 cm⁻¹, 718 cm⁻¹, 862 cm⁻¹, 946 cm⁻¹, 1065 cm⁻¹, 1167 cm⁻¹, 1255 cm⁻¹, and 1375 cm⁻¹ correspond to the presence of boron and some oxides, as shown in FIG. 4A. The band centered at 615 cm⁻¹ may be related to vibration of Sr-O bonds. In contrast, the bands at 715 cm⁻¹ are assigned to the bending vibration B—O—B, the bands located within 800 cm⁻¹ to 1200 cm⁻¹ may be attributed to the B—O stretching of tetrahedral BO₄ unit, while the last bands located between 1200 cm⁻¹ to 1600 cm⁻¹ may be ascribed to the B—O stretching of trigonal BO₃. The immersion of the S0 sample in SBF diminished some bands and reduced the intensity for the remaining bands, indicating a change in the glass structure. The FTIR bands for the sample S0 at 7 days post immersion were 602 cm⁻¹, 715 cm⁻¹, 858 cm⁻¹, 955 cm⁻¹, 1056 cm⁻¹, 1187 cm⁻¹, 1256 cm⁻¹, 1384 cm⁻¹, and 1492 cm⁻¹, while the FTIR spectrum, for the sample S0 at 15 days post immersion, showed bands centered at 602 cm⁻¹, 709 cm⁻¹, 859 cm⁻¹, 944 cm⁻¹, 1044 cm⁻¹, 1044 cm⁻¹, 1054 cm⁻¹, 1161 cm⁻¹, 1392 cm⁻¹, 1510 cm⁻¹. The FTIR bands for the sample S0 at 21 days post immersion were at 602 cm⁻¹, 709 cm⁻¹, 855 cm⁻¹, 964 cm⁻¹, 1153 cm⁻¹, 1287 cm⁻¹, and 1479 cm⁻¹. It may be noted that new bands, after immersion, ranged between 602 cm⁻¹ and 1492 cm⁻¹ to about 1510 cm⁻¹, corresponding to O—P—O bending and the vibration of the phosphate PO₄³⁻, respectively. Further, the FITR results for the S10 sample before immersion in SBF showed several bands located at 723 cm⁻¹, 857 cm⁻¹, 975 cm⁻¹, 1192 cm⁻¹, 1256 cm⁻¹, and 1333 cm⁻¹ corresponding to borate and silicate, as illustrated in FIG. 4B. The first band located at 723 cm⁻¹ may be attributed to bending vibration of B—O—B, while the bands placed between 800 cm⁻¹ to 1200 cm⁻¹ may be attributed to overlapping between the B—O stretching of BO₄ unit and Si—O stretching vibration of SiO₄. The bands located between 1200 cm⁻¹ to 1600 cm⁻¹ correspond to the B—O stretching of trigonal BO₃. The immersion of the S10 sample in SBF shifted and reduced the intensity of bands. In an example, the FTIR band for the sample S10, 7 days post immersion were 602 cm⁻¹, 706 cm⁻¹, 855 cm⁻¹, 974 cm⁻¹, 1107 cm⁻¹, 1255 cm⁻¹, 1330 cm⁻¹, and 1483 cm⁻¹, further, for the sample S10, 15 days post immersion were 602 cm⁻¹, 705 cm⁻¹, 855 cm⁻¹, 968 cm⁻¹, 1153 cm⁻¹, 1288 cm⁻¹, and 1473 cm⁻¹, while the bands for the sample S10, 21 days post immersion were about 602 cm⁻¹, 705 cm⁻¹, 855 cm⁻¹, 958 cm⁻¹, 1153 cm⁻¹, 1276 cm⁻¹, and 1477 cm⁻¹. The bands that appeared at 602 cm⁻¹ and 1477 cm⁻¹ were attributed to O—P—O bending and the vibration of the phosphate PO₄³⁻, respectively. FIG. 4C depicts the FTIR for the sample S20, which showed several bands located at 726 cm⁻¹, 860 cm⁻¹, 918 cm⁻¹, 1188 cm⁻¹, and 1344 cm⁻¹. As mentioned above, the 726 cm⁻¹ is attributed to the bending vibration B—O—B. The band between 800 cm⁻¹ to 1200 cm⁻¹ may be attributed to the overlapping between the B—O stretching of the BO₄ unit and the Si—O stretching vibration of SiO₄. The location of the FTIR bands for the sample S20, 7 days post immersion were at 707 cm⁻¹, 850 cm⁻¹, 966 cm⁻¹, 1208 cm⁻¹, 1344 cm⁻¹, and 1488 cm⁻¹. Further, for the sample S20, 15 days post immersion, the FTIR bands were observed at 726 cm⁻¹, 857 cm⁻¹, 966 cm⁻¹, 1169 cm⁻¹, and 1488 cm⁻¹. In addition, for the sample S20, 21 days post immersion, the FTIR bands were observed at 707 cm⁻¹, 852 cm⁻¹, 966 cm⁻¹, 1140 cm⁻¹, and 1488 cm⁻¹. Post immersion, a band appeared at 1488 cm⁻¹, ascribed to the phosphate PO₄³⁻ vibration. The aforementioned band is an indicator of the formation of hydroxyapatite (HA).

According to the present disclosure, SEM analysis of the three glass sample treated in SBF solution provided insights into the changes in surface morphology of the samples. FIGS. 5A-5C depict the SEM analysis results of the three glass samples. In particular, the SEM at 5 micrometers magnification, reveals the development of apatite on both the surface and the cross-section of discs before and after immersion in SBF for 7 days, 15 days, and 21 days. Referring to FIGS. 5A-5C, the surface of the sample S0 initially appeared smooth and free of crystalline formations. Small crystalline formations appeared after 7 days, increasing in density after 15 days but remaining incomplete. By 21 days, some crystalline formations were present, but the apatite layer was not dense or uniform. Before immersion, the surface is smooth and deposit-free for the sample S10. Post 7 days of immersion, minuscule crystals appear, and their density increased after 15 days, forming a thick, cohesive apatite layer after 21 days. For the sample S20, a similar trend was observed, with small crystalline formations appearing after 7 days, increasing after 15 days, and forming a thick and uniform apatite layer after 21 days of immersion in SBF.

Furthermore, as can be seen from FIGS. 5A-5C, apatite formation was more evident on the surface than in the cross-section of the sample discs. In the samples S10 and S20, dense and uniform apatite layers formed on the surface after 21 days of immersion, while the cross-section shows less dense and uniform formations. The aforementioned observation indicates that surface interactions with SBF contribute more significantly to apatite formation than interactions within the depth of the samples. SEM analysis depicted that the apatite formation gradually developed with increased immersion time in SBF, particularly in samples S10 and S20. Small crystals appeared after 7 days, increasing in density and size over time, and fully covered the surface after 21 days. The aforementioned observation aligns with XRD results for apatite formation on the sample surfaces. Sample S0 shows less clear apatite formation compared to samples S10 and S20.

Table 2 displays the weight loss of the SO, S10, and S20 glass samples as a function of immersion time in the SBF, which was employed to observe the glass kinetic conversion to HA within immersion time of 7 days, 15 days, and 21 days. The weight loss for glass samples showed variation with a change in the B₂O₃ content. For instance, the weight loss values for SO, S10, and S20 samples at seven days were 12.32%, 8.971%, and 10.505%, respectively. The aforementioned values increased with increasing immersion time for up to 15 days, and the values were 26.193%, 11.850%, and 13.760% for SO, S10, and S20, respectively. Post 21 days immersion periods, the weight loss value for S0 was 22.448%, while the weight loss values for S10 and S20 were 12.577% and 15.800%, respectively. In general, silicate has a low degradable rate, and boron has adverse features through high solubility, as mentioned in Table 2. The mix between borate and silicate provided two compositions, S10 and S20, which may be used as bioactive glass. Table 2 shows that the S20 has weight loss higher than S10 sample. The XRD showed the glass transformation to HA by only forming a crystalline phase in S10 and S20. The aforementioned result indicates the bioactivity rate for glass samples, which further indicates the desirable performance of S0 sample, followed by S20 and S10 samples.

TABLE 2
Weight loss of the glass samples S0,
S10, and S20, post immersion in SBF

| Immersion | Glass weight and | Glass codes | | |
|---|---|---|---|---|
| time (days) | weight loss | S0 | S10 | S20 |
| 7 | W0 | 0.560 | 0.535 | 0.495 |
| | Wt | 0.491 | 0.487 | 0.443 |
| | Weight loss (%) | 12.32 | 8.971 | 10.505 |
| 15 | W0 | 0.619 | 0.540 | 0.574 |
| | Wt | 0.438 | 0.476 | 0.495 |
| | Weight loss (%) | 26.193 | 11.850 | 13.760 |
| 21 | W0 | 0.490 | 0.485 | 0.478 |
| | Wt | 0.380 | 0.424 | 0.402 |
| | Weight loss (%) | 22.448 | 12.577 | 15.800 |

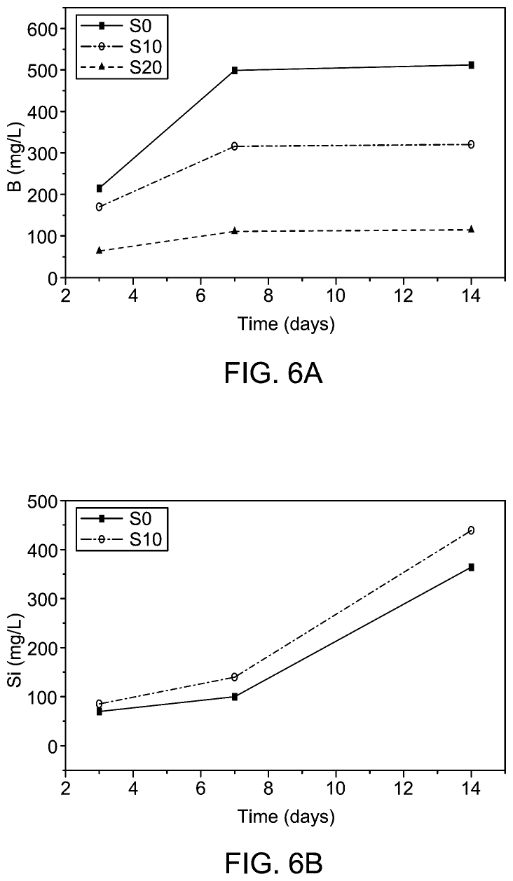

In general, bioactive glass is desired to have an ability to release compositional ions into a surrounding atmosphere and maintain the ion release during dissolution. Increased ion concentrations produce therapeutic reactions like anti-inflammatory, antimicrobial and osteogenic effects. ICP-MS evaluations were undertaken to capture the ion release characteristics of each element in the glass composition. FIGS. 6A-6B depict the Si and B ions release profiles for up to 2 weeks. In particular, FIG. 6A shows the ion release for boron ions for the SO, S10, and S20 samples, and it may be noted that the S0 sample showed the highest boron release due to the high concentration of boron in the S0 sample. Further, FIG. 6B shows the Si ions released from the sample S10 and S20, at a plurality of immersion time. The sample S20 showed the highest Si ions released due to increased Si concentrations. In other words, increased Si contents in glasses showed increased released Si ions, and the sample S20 showed a higher concentration of silicon released in distilled water, followed by the sample S10. The aforementioned observations provides insights into the behavior of materials under different conditions, as it shows how the composition of the material affects the associated dissolution rate and the release of elements into the solution. Further, the evaluations as conducted herein provide insights into the impact of varying element concentrations in the samples and may assist in predicting the performance of materials in various applications, such as medical and engineering applications, thereby improving material design for desirable performance. The results provided herein are compatible with weight loss results, which showed the highest bioactivity for the sample SO compared with the S20 and the S10 samples.

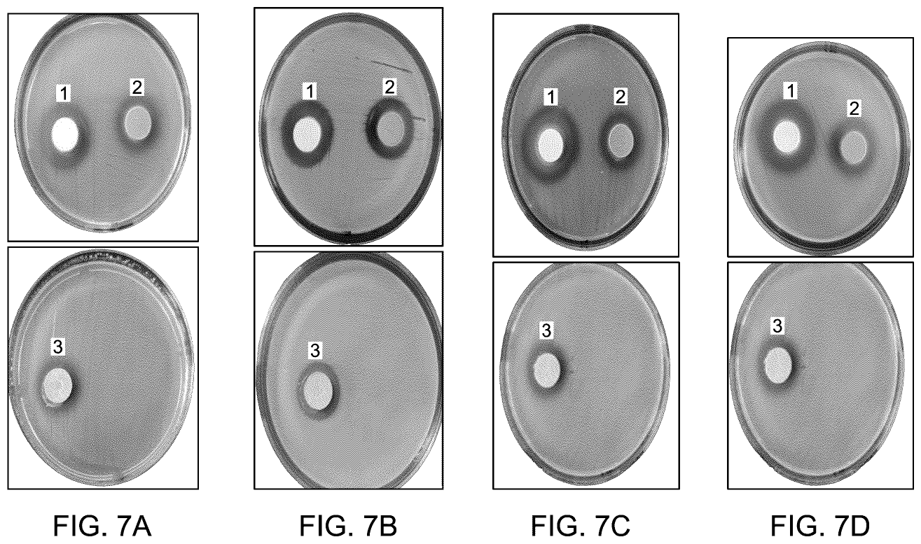

Referring to FIGS. 7A-7D, antibacterial activity of the fabricated samples is illustrated, according to certain aspects. In particular, the antibacterial activity was evaluated using the direct contact method. The fabricated samples showed desirable antibacterial properties against gram-negative bacteria including Escherichia coli (ATCC 25922), Shigella flexneri (ATCC 12022) and gram-positive bacteria including Staphylococcus epidermidis (ATCC 12228) and Staphylococcus aureus (ATCC 29213) with variable degrees of antibacterial activity, as illustrated in Table 3. It may be noted that the maximum antibacterial effect was observed in case of the sample SO, which involves the lowest content of silicon and the highest content of boron. Further, the sample S20 containing the lowest content of boron and the highest content of silicon showed the lowest antibacterial effect. Furthermore, a medium antibacterial effect was observed in of the sample S10, which included a moderate concentration of boron. Hence, increasing boron concentration in the sample formulation favored the associated antibacterial activity. In general, boron-made derivatives and composites have been found to exhibit antimicrobial properties, making boron a favorable agent in various applications, including disinfection and subsequent medical therapeutic applications.

Table 3 furthers depicts that 40% boron concentration was most active against the gram-negative bacteria Shigella flexneri (ATCC 12022) with an inhibition zone diameter of 30.46 mm, and Escherichia coli (ATCC 25922) with the inhibition zone diameter of 29.86 mm. In comparison, the 40% boron concentration exerted lower activity against the gram-positive bacteria Staphylococcus aureus (ATCC 29213) with the inhibition zone diameter of 27.07 mm, and Staphylococcus epidermidis (ATCC 12228) with the inhibition zone diameter of 26.82 mm.

The higher sensitivity of Gram-negative bacteria to boron compounds has been demonstrated earlier [See: Sayin, Zafer, Uckun Sait Ucan, and Asli Sakmanoglu. “Antibacterial and antibiofilm effects of boron on different bacteria], where the gram-positive bacterium Staphylococcus aureus was more resistant while the gram-negative bacteria Vibrio anguillarum and Pseudomonas aeruginosa were the most sensitive. The aforementioned difference in antibacterial activity after exposure to high concentration of boron may be due to the differences in cell wall structures in gram-positive and gram-negative bacteria. Contrary to gram-positive bacteria, the gram-negative bacteria is rich in lipopolysaccharides and poor in mucopeptides and teichoic acids. The lipid-rich molecules were also found to favor interaction with boron [See: Sayin, Z., Ucan, U. S., & Sakmanoglu, A. (2016). Antibacterial and antibiofilm effects of boron on different bacteria]. The boron compounds attach to the cis-hydroxyl groups in cell membranes; hence, they have biological impacts on both humans and animals [See: Kabu, Mustafa, Cangir Uyarlar, Katarzyna Zarczynska, Wanda Milewska, and Przemyslaw Sobiech. “The role of boron in animal health.”]. Boron is necessary for humans and animals, even at lower doses. However, dose-dependent side effects of some boron compounds were observed in research [See: Hernandez, Vincent, Thibaut Crépin, Andrés Palencia, Stephen Cusack, Tsutomu Akama, Stephen J. Baker, Wei Bu et al. “Discovery of a novel class of boron-based antibacterials with activity against gram-negative bacteria]. Although boron has been known to have antibacterial properties against numerous fungi and bacteria, it is unknown what impact boron has on other microbes.

TABLE 3
Effect of bioactive glass composition on antibacterial activity

| | Antibacterial activity (mm) | | |
|---|---|---|---|
| Tested microbe | S0 | S10 | S20 |
| Staphylococcus epidermidis ATCC 12228 | 26.82 | 23.54 | 21.63 |
| Staphylococcus aureus ATCC 29213 | 27.07 | 24.94 | 20.28 |
| Escherichia coli ATCC 25922 | 29.86 | 22.04 | 21.65 |
| Shigella flexneri ATCC 12022 | 30.46 | 23.05 | 20.61 |

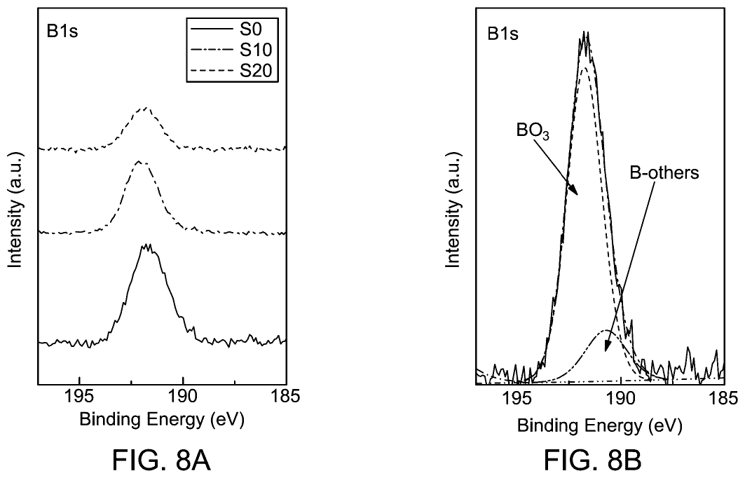

XPS was used to explore the structure of the three glass samples. FIG. 8A shows the high-resolution core level B 1s spectra in the binding energy (BE) range of 197 electron volts (eV) to 185 eV. A peak at a BE of about 192 eV with a full width at half maximum (FWHM) of about 2 eV for all samples was observed. No shift has been recorded for the B 1s spectra. Further, FIG. 8B displays the fitting of the B 1s core level spectrum for sample SO. The core level spectrum is fitted with two contributions, a strong peak at a BE of 191.8 eV, FWHM of 1.98 eV and a small peak at a BE of 190.7 eV, FWHM of 2.33 eV. Hence, it may be concluded that boron atoms may be in two different molecular environments in this glass, BO₃ with a percentage of about 84% and (B—Sr, B—Na, B—Si, B—Ca) with a percentage of about 16%, as listed in Table 4 [See: Meng, Xianglong, Bing Xiao, and Hengheng Wu. “Effects of Ti containing Cu-based alloy on sintering mechanism, element diffusion behaviour and physical properties of glass-ceramic bond for cubic boron nitride abrasive tool materials, and Mhareb, M. H. A., Abdelkrim Mekki, Amnah Alwabsi, Ashwag Almaimouni, Hammam Abdurabu Thabit, Nouf Alshwaira, Istbrq Ahmad Alsaleh, and Fatimah Awn Al-Dhahi. “X-ray photoelectron spectroscopy, structural, and radiation shielding properties for transparent borosilicate glasses]. The samples S10 and S20 were fitted with two contributions similar to the fitting of the SO sample. The results of the fitting are listed in Table 4. It may be concluded that BO₃ content increased from 84% to 95% with a rise in SiO₂ concentration in the glass.

TABLE 4
Fitting parameters of B 1s core level spectra

| | B1s (BO3) | | | B1s (B-others) | | |
|---|---|---|---|---|---|---|
| B1s | BE (eV) | FWHM (eV) | % | BE (eV) | FWHM (eV) | % |
| S0 | 191.8 | 1.98 | 84 | 190.7 | 2.33 | 16 |
| S10 | 192.5 | 1.75 | 90 | 190.6 | 2.41 | 10 |
| S20 | 192.5 | 2.01 | 95 | 190.4 | 2.35 | 5 |

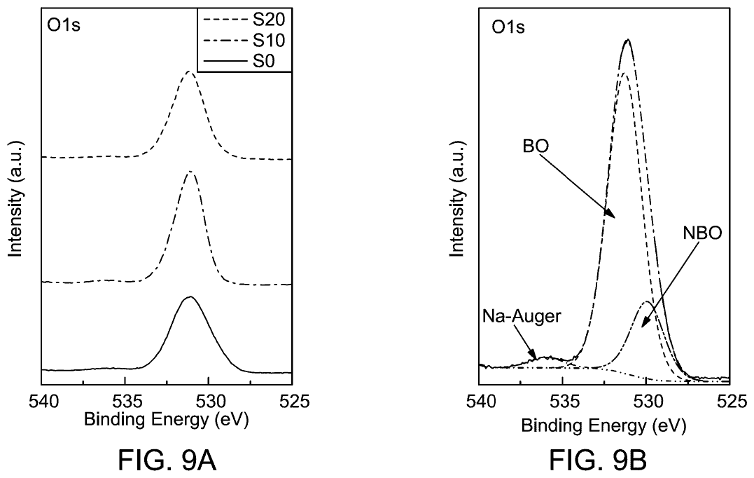

The oxygen molecular environment in the three glass samples was investigated by recording the XPS O 1s core level spectra of the three glass samples. FIG. 9A shows the spectra in the BE range of 540 eV to 525 eV. As can be seen from FIG. 9A, a peak was observed at a BE of about 531 eV. Further, no shift in the BE of the three spectra was observed. A small peak at about 536 eV was due to Na-Auger.

In general, oxygen in glasses exist in two coordination geometries, namely non-bridging oxygen (NBO) and bridging oxygen (BO) [See: Lai, Feifei, Wen Yao, and Jiangling Li. “Effect of B2O3 on structure of CaO—Al₂O₃—SiO₂—TiO₂—B₂O₃ glassy systems]. Therefore, in the present disclosure, the O 1s spectra was fitted with two contributions, one due to NBO and other due to BO. Boron and silicon are glass former contributing to the BO sites, and sodium is a glass modifier, disrupting the glass network and contributing to the NBO. Therefore, the BO sites are due to oxygen in B—O—B, Si—O—Si, and B—O—Si, while NBO sites are due to oxygen in B—O—Na and Si—O—Na. Further, Sr and Ca are mainly intermediate glass. Depending on their concentrations in the glass network, Sr and Ca contribute to either BO or NBO sites. FIG. 9B shows the fitting of O 1s for sample S0 with two peaks. A first peak was due to BO at a BE of 531.3 eV and FWHM of 2.42 eV, and a second peak was due to NBO at a BE of 529.8 eV. The concentration of BO is 80.3%, while the NBO is 19.7%. The glass has a high concentration of BO; hence the glass network is stable. Table 5 provides a summary of the O 1s spectra fitting results. It may be concluded that replacing boron oxide with silicon oxide increases the number of BO in the glass, rendering the glass more stable.

TABLE 5
Fitting parameter of O 1s core level spectra

| | O 1s (BO) | | | O1s (NBO) | | |
|---|---|---|---|---|---|---|
| O1s | BE (eV) | FWHM (eV) | % | BE (eV) | FWHM (eV) | % |
| S0 | 531.3 | 2.42 | 80.3 | 529.8 | 2.30 | 19.7 |
| S10 | 532.0 | 1.79 | 85.5 | 530.0 | 2.00 | 14.5 |
| S20 | 531.5 | 2.35 | 100 | — | — | — |

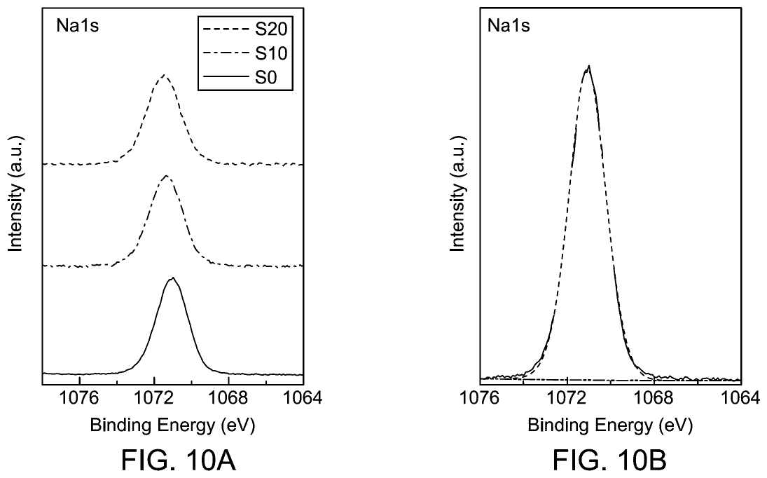

FIG. 10A shows the high-resolution Na 1s spectra for the three glass samples. The spectra are symmetric with a peak of BE at about 171 eV. Further, the BE showed a small shift toward higher BE from 171.0 eV for the sample S0 to 171.4 eV for the sample S20. The observed slight increase in BE energy may be attributed to the rise in Si concentration in the glass. FIG. 10B displays the fitting of the Na 1s spectrum for sample SO. Similar single peak fitting was carried out for the other two samples. The FWHM of the fitted peak was found to be 2.24 eV. The results indicate that Na ions are in the Na⁺ oxidation state.

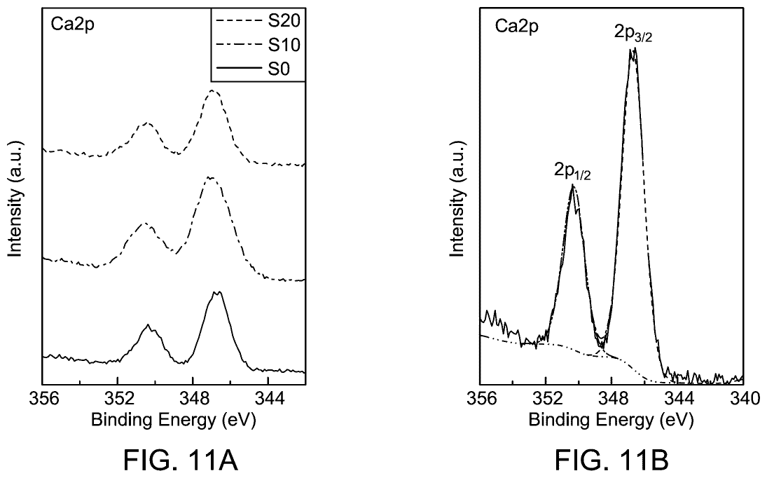

FIG. 11A shows the high-resolution Ca 2p spectra for the glasses. The spectra included spin-orbit doublet of the 2p level, further including 2p_{3/2} and 2p_{1/2}. The BE of Ca 2p_{1/2} was about 350 eV, and about 365 eV for Ca 2p_{3/2} with an energy separation of about 4 eV. FIG. 11B shows the curve fitting of Ca 2p for the sample SO. The 2p_{3/2} peak has a BE of 346.8 eV, and the 2p_{1/2} peak had a BE of about 350.4 eV with an energy separation of about 3.6 eV. A similar fitting was performed on the other samples and the results are summarized in Table 6. As can be seen from Table 6, no significant shift in the prominent peak was observed with the insertion of SiO₂ in the glass matrix. The energy separation between the spin-orbit doublet and BE values indicate that Ca exits in the Ca²⁺ oxidation state [See: Khattak, G. D., A. Mekki, and M. A. Gondal. XPS studies of pulsed laser induced surface modification of vanadium phosphate glass samples]. Furthermore, the spin-orbit area ratio found in the present disclosure agrees with the theoretical value of 1:2 for all samples.

TABLE 6
Fitting parameters of Ca 2p core level spectra

| | Ca2p_{3/2} | | | Ca2p_{1/2} | | | |
|---|---|---|---|---|---|---|---|
| | BE | FWHM | | BE | FWHM | | 2p_{3/2} − |
| Ca2p | (eV) | (eV) | % | (eV) | (eV) | % | 2p_{1/2} (eV) |
| S0 | 346.8 | 1.91 | 66 | 350.4 | 1.78 | 34 | 3.6 |
| S10 | 347.0 | 1.60 | 66 | 350.6 | 1.60 | 34 | 3.6 |
| S20 | 346.8 | 1.72 | 66 | 350.5 | 1.72 | 34 | 3.7 |

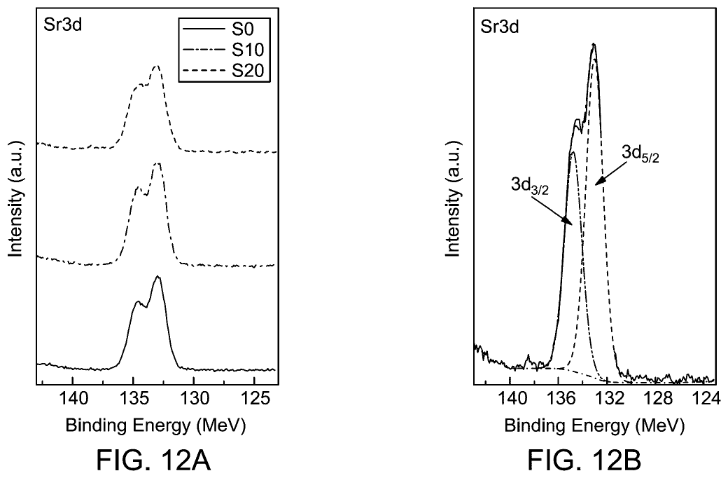

The Sr 3d core level spectra for the three glasses are shown in FIG. 12A. As can be seen from FIG. 12A, the 3D core-level spectra have a typical spin-orbit doublet. An energy of separation for the spin-orbit doublet was around 2 eV, and the BE of 3d_{1/2} peak was about 135 eV, while 3d_{5/2} peak is approximately 133 eV. The peaks were curve-fitted to get accurate values of the BE and the FWHM. FIG. 12B shows the fitting of the sample S20. The 2p_{3/2} peak had a BE of 133.0 eV, while the BE for 2p_{1/2} peak was about 134.8 eV with an energy separation of 1.8 eV. The samples S0 and S10 were fitted in a similar manner. The results of the fitting are summarized in Table 7. It may be concluded that Sr atoms may be available in different bonds including Sr—O—Si, Sr—O—B, Sr—O—Na and Sr—O—Ca. The table shows that the spin-orbit area ratio found in the present disclosure agrees with the theoretical value of 2:3 for all samples.

TABLE 7
Fitting parameters of Sr 3d core level spectra

| | Sr 3d_{5/2} | | | Sr 3d_{3/2} | | | |
|---|---|---|---|---|---|---|---|
| | BE | FWHM | | BE | FWHM | | 3d_{5/2} − |
| Sr 3d | (eV) | (eV) | % | (eV) | (eV) | % | 3d_{3/2} (eV) |
| S0 | 133.1 | 1.80 | 59 | 134.8 | 1.80 | 41 | 1.7 |
| S10 | 133.0 | 1.56 | 59 | 134.7 | 1.56 | 41 | 1.7 |
| S20 | 133.0 | 1.68 | 59 | 134.8 | 1.68 | 41 | 1.8 |

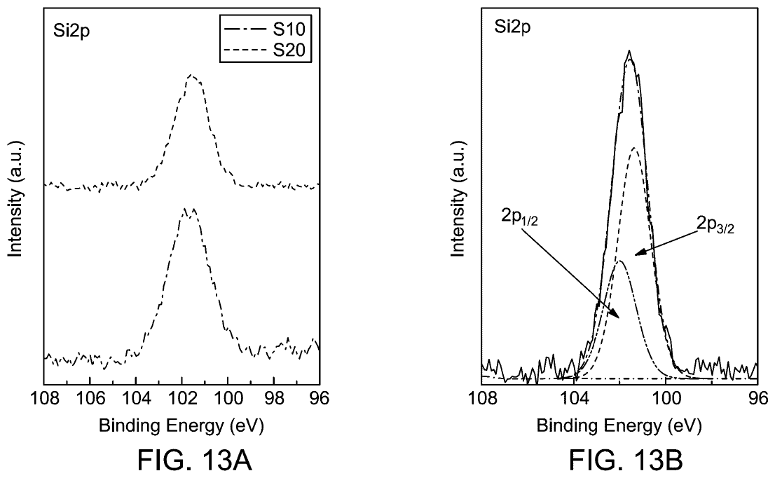

The Si 2p core level spectra for the samples S10 and S20 stacked together are shown in FIG. 13A. In particular, FIG. 13A shows symmetric peaks with a BE of about 101.5 eV for the sample S10 and a BE of about 101.7 eV for the S20 sample and an FWHM of about 2.3 eV for both spectra in agreement with previously published research for copper-sodium silicate glasses [See: Mhareb, M. H. A., Abdelkrim Mekki, Amnah Alwabsi, Ashwag Almaimouni, Hammam Abdurabu Thabit, Nouf Alshwaira, Istbrq Ahmad Alsaleh, and Fatimah Awn Al-Dhahi. X-ray photoelectron spectroscopy, structural, and radiation shielding properties for transparent borosilicate glasses; Jain, Nishant, Johannes Schmidt, Oliver Görke, David Karl, Aleksander Gurlo, and Franziska Schmidt. “Surface modification of bioactive glasses for successful incorporation with poly (lactic-co-glycolic acid) (PLGA); and Sawyer, R., H. W. Nesbitt, and R. A. Secco. High resolution X-ray photoelectron spectroscopy (XPS) study of K₂O—SiO₂ glasses: Evidence for three types of O and at least two types of Si]. A slight shift toward higher BE was observed with increased SiO₂ concentration in the glass matrix. The aforementioned result indicates a noticeable change in the local structure around the Si atom in the glass matrix when the concentration of SiO₂ was increased from 10 mol. % to 20 mol. %. The change in the local structure may be confirmed by curve fitting the two spectra. FIG. 13B displays the curve fitting of the Si 2p spectra for the sample S10. The 2p_{1/2} and 2p_{3/2} spin-orbit doublet was used to suit the peak. The best fit was obtained when the area ratio of the two peaks was 0.51, close to the theoretical value of 1:2 for the 2p peaks. Table 8 depicts an overview of the outcomes of fitting the two spectra. The BE for the 2p_{1/2} peak and the 2p_{3/2} peak for the sample S10 are 102.1 eV and 101.5 eV, while the BE for the 2p_{3/2} peak and the 2p_{1/2} peak for the sample S20 was about 103.0 eV and 102.2 eV, respectively. The BE of SiO₂ was about 103.85 eV [See: Nesbitt, H. W., G. M. Bancroft, G. S. Henderson, R. Ho, K. N. Dalby, Y. Huang, and Z. Yan. “Bridging, non-bridging and free (O2−) oxygen in Na2O—SiO2 glasses: An X-ray photoelectron spectroscopic (XPS) and nuclear magnetic resonance (NMR) study]. The aforementioned values indicate that Si does not have the SiO₂ coordination geometry

TABLE 8
Fitting parameters of the Si 2p core level spectra

| | Si 2p_{3/2} | | | Si 2p_{1/2} | | | |
|---|---|---|---|---|---|---|---|
| | BE | FWHM | | BE | FWHM | | 2p_{3/2} − |
| Si 2p | (eV) | (eV) | % | (eV) | (eV) | % | 2p_{1/2} (eV) |
| S10 | 101.5 | 2.09 | 66.2 | 102.1 | 2.09 | 33.8 | 0.6 |
| S20 | 102.2 | 1.75 | 66.2 | 103.0 | 1.75 | 33.8 | 0.8 |

The aspects of the present disclosure provide the bioglass composition in the form of a particle. In particular, the effects of varying concentration of SiO₂ and B₂O₃ on the formation of hydroxyapatite (HA) in glass samples when immersed in simulated body fluid (SBF) was evaluated and examined. The three different glass compositions (SO, S10, and S20) were prepared with varying ratios of SiO₂ and B₂O₃ while keeping the calcium, sodium, and strontium concentrations constant. The XRD analysis initially indicated an amorphous structure for all glasses before immersion. However, after 7 days, crystallization and HA formation became apparent, with further progression observed by day 21 for the samples S10 and S20. The influence of boron on crystallization was observed, affecting the formation of HA, and the XRD results for S0 showed an absence of crystalline peaks for HA with immersion, indicating non-biocompatibility for the S0 sample. The FTIR analysis revealed changes in the functional groups and chemical bonds of the glass samples after immersion in SBF, indicating the formation of HA. The specific peaks and subsequent intensities varied depending on the composition of the samples, particularly the ratios of SiO₂ and B₂O₃. The aforementioned findings provide insights into the chemical interactions and reactions occurring during the immersion process, contributing to a better understanding of the bioactivity and behavior of the prepared glass compositions in simulated physiological conditions. The antibacterial efficacy of the samples was evaluated, yielding favorable outcomes, particularly in the presence of boron, thus rendering it valuable for medical and therapeutic implementations. The S0 sample showed the highest weight loss during immersion in SBF due to a high amount of B₂O₃, followed by the S20 and the S10 samples. The result aligns with the ion concentration released, as evaluated by the ICP instrument. XPS was used to analyze the local structure, oxidation state, and coordination geometry of the various elements in the prepared glass samples. Oxygen ions were found to be predominantly in the BO environment. The Ca ions were found to be in the Ca²⁺ oxidation state in the CaO environment. The Na ions were found to be in the Na⁺ oxidation state, that is, the Na₂O environment. Sr ions were found in the certain environments including Sr—O—Sr, Sr—O—B, Sr—O—Si, Sr—O—Na, and Sr—O—Ca. Si ions were found in SiO₃ and/or SiO₄ chemical environments. Boron ions were found to exist predominantly in the BO₃ chemical environment. Based on the above results, the S10 and S20 samples showed high biocompatibility and bioactivity, which are desirable products for use as biomaterials in medical therapeutic applications.

Numerous modifications and variations of the present disclosure are possible in light of the above teachings. It is therefore to be understood that within the scope of the appended claims, the invention may be practiced otherwise than as specifically described herein.

1. A bioglass composition in the form of a particle, having:
a core portion, a crystalline zone and a surface layer,
wherein the surface layer is hydroxyapatite,
wherein the crystalline layer is beneath the surface layer and surrounds the core portion; and
wherein the bioglass composition comprises:
10-20 mol. % SiO₂;
20 mol. % SrO;
20 mol. % Na₂O;
20 mol. % CaO; and
30-40 mol. % B₂O₃.

2. The composition of claim 1, in the form of a pressed pellet comprising a plurality of particles of the composition, wherein the particles have a longest dimension in a range from 0.5 to 1000 mm.

3. The composition of claim 2, wherein the pressed pellet has a circular form with a circumference in a range from 0.5 to 100 mm and a thickness in a range from 0.1 to 100 mm.

4. The composition of claim 1, wherein the surface layer includes hydroxyapatite crystals formed by immersion in a simulated body fluid.

5. The composition of claim 4, wherein hydroxyapatite crystals are in the form of a uniform layer and are formed by 21 days immersion in the simulated body fluid.

6. The composition of claim 1, wherein the bioglass composition comprises 10 mol % SiO₂, 20 mol % SrO, 20 mol % Na₂O, 20 mol % CaO, and 40 mol % B₂O₃ and has a weight loss greater than or equal to 8 percent after 7 days of immersion in a simulated body fluid.

7. The composition of claim 6, wherein the bioglass composition has a weight loss greater than or equal to 10 percent after 15 days of immersion in simulated body fluid.

8. The composition of claim 7, wherein the bioglass composition has a weight loss greater than or equal to 12 percent after 21 days of immersion in simulated body fluid.

9. The composition of claim 1, wherein the bioglass composition comprises 20 mol % SiO₂, 20 mol % SrO, 20 mol % Na₂O, 20 mol % CaO, and 30 mol % B₂O₃ and has a weight loss greater than or equal to 10 percent after 7 days of immersion in simulated body fluid.

10. The composition of claim 9, wherein the bioglass composition has a weight loss greater than or equal to 13 percent after 15 days of immersion in simulated body fluid.

11. The composition of claim 10, wherein the bioglass composition has a weight loss greater than or equal to 15 percent after 21 days of immersion in simulated body fluid.

12. The composition of claim 1, wherein X-ray diffraction shows evidence of calcium deficient hydroxyapatite formation by the presence of a peak at around 26° after 21 days of immersion in simulated body fluid.

13. The composition of claim 6, wherein the bioglass composition has an antibacterial activity of greater than or equal to 23 mm for Staphylococcus epidermidis when measured in a direct contact incubation test.

14. The composition of claim 6, wherein the bioglass composition has an antibacterial activity of greater than or equal to 24 mm for Staphylococcus aureus when measured in a direct contact incubation test.

15. The composition of claim 6, wherein the bioglass composition has an antibacterial activity of greater than or equal to 21 mm for Escherichia coli when measured in a direct contact incubation test.

16. The composition of claim 6, wherein the bioglass composition has an antibacterial activity of greater than or equal to 22 mm for Shigella flexneri when measured in a direct contact incubation test.

17. The composition of claim 9, wherein the bioglass composition has an antibacterial activity of greater than or equal to 21 mm for Staphylococcus epidermidis when measured in a direct contact incubation test.

18. The composition of claim 9, wherein the bioglass composition has an antibacterial activity of greater than or equal to 20 mm for Staphylococcus aureus when measured in a direct contact incubation test.

19. The composition of claim 9, wherein the bioglass composition has an antibacterial activity of greater than or equal to 21 mm for Escherichia coli when measured in a direct contact incubation test.

20. The composition of claim 9, wherein the bioglass composition has an antibacterial activity of greater than or equal to 20 mm for Shigella flexneri when measured in a direct contact incubation test.
